(19) United States
(12) Patent Application Publication
(10) Pub. No.: US 2025/0110127 A1
(43) Pub. Date: Apr. 3, 2025

(54) USE AND DETECTION METHOD OF FLAVIVIRUS PROTEIN MICROARRAY

(71) Applicant: NATIONAL CHENG KUNG UNIVERSITY, Tainan City (TW)

(72) Inventors: Guan-da SYU, Tainan City (TW); Tzong-shiann HO, Tainan City (TW)

(21) Appl. No.: 18/480,009

(22) Filed: Oct. 3, 2023

(51) Int. Cl. G01N 33/569; G01N 33/543

(52) U.S. Cl. CPC G01N 33/56983; G01N 33/54306; G01N 2333/185; G01N 2469/20; G01N 2470/04; G01N 2800/56

(57) ABSTRACT

A use and detection method of flavivirus protein microarray are provided. The flavivirus protein microarray having an amino acid sequence of SEQ ID NO: 1, an amino acid sequence of SEQ ID NO: 3, and an amino acid sequence of SEQ ID NO: 13 may be used to distinguish dengue patients with mild case from hospitalized case. The flavivirus protein microarray having an amino acid sequence of SEQ ID NO: 3 and an amino acid sequence of SEQ ID NO: 4 may be used to distinguish dengue patients with mild case and hospitalized non-severe case from hospitalized severe case. The method for distinguishing dengue patients with mild case from hospitalized case comprises steps of providing the flavivirus protein microarray, subsequently adding a non-protein blocking reagent, a serum of the dengue patient, and a fluorescently labeled anti-human immunoglobulin antibody to the flavivirus protein microarray and reading an optical signal.

### INCORPORATION BY REFERENCE OF SEQUENCE LISTING PROVIDED AS A XML FILE

The sequence listing submitted via EFS, in compliance with 37 CFR 1.52 (e) (5), is incorporated herein by reference. The sequence listing XML file contains the replacement file TP203615-US-SEQUENCELIST.xml, created on Nov. 7, 2023 which is 24,651 bytes in size.

### FIELD OF INVENTION

The present disclosure relates to the technical field of protein microarray, and particularly, to a flavivirus protein microarray; the present disclosure also relates to the technical field of a use, and particularly, to a use of the flavivirus protein microarray; and the present disclosure also relates to the technical field of a detection method, and particularly, to a detection method of the flavivirus protein microarray.

### BACKGROUND OF INVENTION

Dengue Fever (DF) is caused by the infection of dengue virus (DENV). Dengue virus belongs to the family Flaviviridae, genus Flavivirus composed of three structural proteins (capsid, membrane, and envelope protein) and seven nonstructural proteins (NS1, NS2A, NS2B, NS3, NS4A, NS4B, and NS5 protein). The other Flaviviridae family that infected humans also showed 50-60% similarity of the envelope proteins, including Zika virus, Japanese encephalitis virus, West Nile virus, Yellow fever virus, and Tick-borne Encephalitis virus.

Dengue virus infection may be asymptomatic or cause severe complications. After infection with one serotype, it will be immune to the serotype for life. However, subsequent infection with other serotypes will increase the risk of severe disease and even death. According to the latest classification of the WHO in 2009, dengue patients are divided into three groups, mild case (group A), hospitalized non-severe case (group B), and hospitalized severe (or ICU) case (group C). In the hospitalized severe cases, there are many serious complications with 40%-50% lethality, e.g., dengue hemorrhagic fever and dengue shock syndrome. Early detection and access to appropriate medical care can reduce the mortality rate of severe dengue to less than 1%.

The conventional technology mainly utilizes polymerase chain reaction (PCR) to determine whether a subject is infected by dengue virus. In other words, PCR can be used to distinguish dengue patients from healthy subjects. However, PCR cannot be used to distinguish dengue patients with mild case (group A) from hospitalized non-severe case (group B) or hospitalized severe case (group C). Moreover, hospitalized severe case is difficult to predict in the clinic, as patients with severe symptoms usually occur after symptoms subside. Furthermore, since the major cause of hospitalized severe case is the antibody cross-reactivity and there is no molecule marker for severity prediction to date, prediction of hospitalized severe case in early diagnosis is challenging. Thus, the conventional technology may not predict that whether a dengue patient with mild case will develop into hospitalized non-severe case or hospitalized severe case for reducing mortality.

Therefore, development of a high-sensitivity and high-throughput flavivirus protein microarray that may distinguish dengue patients with mild case (group A) from hospitalized non-severe case (group B) or from hospitalized severe case (group C) and predict that whether a dengue patient with mild case will develop into hospitalized non-severe case or hospitalized severe case for reducing mortality is an urgent problem to be solved in the art.

### SUMMARY OF INVENTION

To solve the problems mentioned above, one object of the present disclosure is to provide a use of a flavivirus protein microarray for distinguishing dengue patients with mild case from hospitalized non-severe case and hospitalized severe case.

Another object of the present disclosure is to provide a use of a flavivirus protein microarray for distinguishing dengue patients with mild case and hospitalized non-severe case from hospitalized severe case.

Still another object of the present disclosure is to provide a method for distinguishing dengue patients with mild case from hospitalized non-severe case and hospitalized severe case.

In order to achieve the objects mentioned above, the present disclosure provides a flavivirus protein microarray. The flavivirus protein microarray comprises:
a substrate including a plurality of protein array blocks on a surface of the substrate; and
at least one protein immobilized on each of the plurality of protein array blocks, wherein the at least one protein comprises an amino acid sequence of SEQ ID NO: 1, an amino acid sequence of SEQ ID NO: 3, and an amino acid sequence of SEQ ID NO: 13.

In one embodiment, the at least one protein further comprises an amino acid sequence of SEQ ID NO: 4, an amino acid sequence of SEQ ID NO: 5, an amino acid sequence of SEQ ID NO: 8, an amino acid sequence of SEQ ID NO: 12, an amino acid sequence of SEQ ID NO: 13, an amino acid sequence of SEQ ID NO: 16, or any combination thereof.

In one embodiment, the at least one protein further comprises an amino acid sequence of SEQ ID NO: 2, an amino acid sequence of SEQ ID NO: 6, an amino acid sequence of SEQ ID NO: 10, an amino acid sequence of SEQ ID NO: 14, an amino acid sequence of SEQ ID NO: 15, or any combination thereof.

The present disclosure further provides a use of a flavivirus protein microarray for distinguishing dengue patients with mild case from hospitalized non-severe case and hospitalized severe case. The hospitalized case comprises hospitalized non-severe case and hospitalized severe case. The flavivirus protein microarray comprises:
a substrate including a plurality of protein array blocks on a surface of the substrate; and
at least one protein immobilized on each of the plurality of protein array blocks, wherein the at least one protein comprises an amino acid sequence of SEQ ID NO: 1, an amino acid sequence of SEQ ID NO: 3, and an amino acid sequence of SEQ ID NO: 13, and
wherein a serum IgM of the dengue patients against the amino acid sequence of SEQ ID NO: 1 is used to distinguish the dengue patients with mild case from the hospitalized non-severe case, a serum IgG of the dengue patients against the amino acid sequence of SEQ ID NO: 3 is used to distinguish the dengue patients with mild case from the hospitalized severe case, and the serum IgG of the dengue patients against the amino acid sequence of SEQ ID NO: 13 is used to distinguish the dengue patients with mild case from the hospitalized case.

In one embodiment, a combination of the serum IgM of the dengue patients against the amino acid sequence of SEQ ID NO: 1 and the serum IgG of the dengue patients against the amino acid sequence of SEQ ID NO: 3 is used to distinguish the dengue patients with mild case from the hospitalized non-severe case.

In one embodiment, the at least one protein further comprises an amino acid sequence of SEQ ID NO: 4, and wherein a combination of the serum IgG of the dengue patients against the amino acid sequence of SEQ ID NO: 3 and the serum IgM of the dengue patients against the amino acid sequence of SEQ ID NO: 4 is used to distinguish the dengue patients with mild case from the hospitalized severe case.

In one embodiment, the at least one protein further comprises an amino acid sequence of SEQ ID NO: 16, and wherein a combination of the serum IgG of the dengue patients against the amino acid sequence of SEQ ID NO: 13 and the serum IgG of the dengue patients against the amino acid sequence of SEQ ID NO: 16 is used to distinguish the dengue patients with mild case from the hospitalized case.

In one embodiment, the at least one protein further comprises an amino acid sequence of SEQ ID NO: 12, and wherein a combination of the serum IgM of the dengue patients against the amino acid sequence of SEQ ID NO: 1, the serum IgG of the dengue patients against the amino acid sequence of SEQ ID NO: 3, and the serum IgG of the dengue patients against the amino acid sequence of SEQ ID NO: 12 is used to distinguish the dengue patients with mild case from the hospitalized non-severe case.

In one embodiment, a combination of the serum IgG of the dengue patients against the amino acid sequence of SEQ ID NO: 3, the serum IgM of the dengue patients against the amino acid sequence of SEQ ID NO: 4, and the serum IgG of the dengue patients against the amino acid sequence of SEQ ID NO: 4 is used to distinguish the dengue patients with mild case from the hospitalized severe case.

In one embodiment, the at least one protein further comprises an amino acid sequence of SEQ ID NO: 8, a combination of the serum IgG of the dengue patients against the amino acid sequence of SEQ ID NO: 13, the serum IgG of the dengue patients against the amino acid sequence of SEQ ID NO: 16, and the serum IgM of the dengue patients against the amino acid sequence of SEQ ID NO: 8 is used to distinguish the dengue patients with mild case from the hospitalized case.

In one embodiment, the at least one protein further comprises an amino acid sequence of SEQ ID NO: 5 and an amino acid sequence of SEQ ID NO: 12, and wherein a combination of the serum IgG of the dengue patients against the amino acid sequence of SEQ ID NO: 5 and the serum IgG of the dengue patients against the amino acid sequence of SEQ ID NO: 12 is used to distinguish the dengue patients with mild case from the hospitalized case.

In one embodiment, the at least one protein further comprises an amino acid sequence of SEQ ID NO: 4; and wherein a combination of the serum IgG of the dengue patients against the amino acid sequence of SEQ ID NO: 5, the serum IgG of the dengue patients against the amino acid sequence of SEQ ID NO: 12, and the serum IgM of the dengue patients against the amino acid sequence of SEQ ID NO: 4 is used to distinguish the dengue patients with mild case from the hospitalized case.

In one embodiment, the at least one protein further comprises an amino acid sequence of SEQ ID NO: 2, an amino acid sequence of SEQ ID NO: 4, an amino acid sequence of SEQ ID NO: 5, an amino acid sequence of SEQ ID NO: 10, an amino acid sequence of SEQ ID NO: 12, an amino acid sequence of SEQ ID NO: 14, or any combination thereof, and wherein the serum IgG of the dengue patients against the amino acid sequence of SEQ ID NO: 1, serum IgG of the dengue patients against the amino acid sequence of SEQ ID NO: 2, the serum IgG of the dengue patients against the amino acid sequence of SEQ ID NO: 4, the serum IgG of the amino acid sequence of SEQ ID NO: 5, the serum IgG of the dengue patients against the amino acid sequence of SEQ ID NO: 10, the serum IgG of the dengue patients against the amino acid sequence of SEQ ID NO: 12, the serum IgG of the dengue patients against the amino acid sequence of SEQ ID NO: 14, or any combination thereof is used to distinguish the dengue patients with mild case from the hospitalized non-severe case.

In one embodiment, the at least one protein further comprises an amino acid sequence of SEQ ID NO: 6, and wherein the serum IgM of the amino acid sequence of SEQ ID NO: 2, the serum IgM of the dengue patients against the amino acid sequence of SEQ ID NO: 4, the serum IgM of the amino acid sequence of SEQ ID NO: 5, the serum IgM of the amino acid sequence of SEQ ID NO: 6, or any combination thereof is used to distinguish the dengue patients with mild case from the hospitalized non-severe case.

In one embodiment, the at least one protein further comprises an amino acid sequence of SEQ ID NO: 4, an amino acid sequence of SEQ ID NO: 5, an amino acid sequence of SEQ ID NO: 8, an amino acid sequence of SEQ ID NO: 10, an amino acid sequence of SEQ ID NO: 12, an amino acid sequence of SEQ ID NO: 14, an amino acid sequence of SEQ ID NO: 15, or any combination thereof, and wherein the serum IgG of the dengue patients against the amino acid sequence of SEQ ID NO: 1, the serum IgG of the dengue patients against the amino acid sequence of SEQ ID NO: 4, the serum IgG of the dengue patients against the amino acid sequence of SEQ ID NO: 5, the serum IgG of the dengue patients against the amino acid sequence of SEQ ID NO: 8, the serum IgG of the dengue patients against the amino acid sequence of SEQ ID NO: 10, the serum IgG of the dengue patients against the amino acid sequence of SEQ ID NO: 12, the serum IgG of the dengue patients against the amino acid sequence of SEQ ID NO: 14, the serum IgG of the dengue patients against the amino acid sequence of SEQ ID NO: 15, or any combination thereof is used to distinguish the dengue patients with mild case from the hospitalized severe case.

In one embodiment, the serum IgM of the dengue patients against the amino acid sequence of SEQ ID NO: 4 is used to distinguish the dengue patients with mild case from the hospitalized severe case.

In one embodiment, the at least one protein further comprises an amino acid sequence of SEQ ID NO: 2, an amino acid sequence of SEQ ID NO: 4, an amino acid sequence of SEQ ID NO: 5, an amino acid sequence of SEQ ID NO: 10, an amino acid sequence of SEQ ID NO: 12, an amino acid sequence of SEQ ID NO: 14, an amino acid sequence of SEQ ID NO: 15, or any combination thereof, and wherein the serum IgG of the dengue patients against the amino acid sequence of SEQ ID NO: 1, the serum IgG of the dengue patients against the amino acid sequence of SEQ ID NO: 2, the serum IgG of the dengue patients against the amino acid sequence of SEQ ID NO: 4, the serum IgG of the dengue patients against the amino acid sequence of SEQ ID NO: 5, the serum IgG of the dengue patients against the amino acid sequence of SEQ ID NO: 10, the serum IgG of the dengue patients against the amino acid sequence of SEQ ID NO: 12, the serum IgG of the dengue patients against the amino acid sequence of SEQ ID NO: 14, the serum IgG of the dengue patients against the amino acid sequence of SEQ ID NO: 15, or any combination thereof is used to distinguish the dengue patients with mild case from the hospitalized case.

In one embodiment, the serum IgM of the dengue patients against the amino acid sequence of SEQ ID NO: 4 is used to distinguish the dengue patients with mild case from the hospitalized case.

In one embodiment, the at least one protein further comprises an amino acid sequence of SEQ ID NO: 2, an amino acid sequence of SEQ ID NO: 4, an amino acid sequence of SEQ ID NO: 5, an amino acid sequence of SEQ ID NO: 10, an amino acid sequence of SEQ ID NO: 12, an amino acid sequence of SEQ ID NO: 14, an amino acid sequence of SEQ ID NO: 15, or any combination thereof, and wherein the serum IgG of the dengue patients against the amino acid sequence of SEQ ID NO: 2, the serum IgG of the dengue patients against the amino acid sequence of SEQ ID NO: 4, the serum IgG of the dengue patients against the amino acid sequence of SEQ ID NO: 5, the serum IgG of the dengue patients against the amino acid sequence of SEQ ID NO: 10, the serum IgG of the dengue patients against the amino acid sequence of SEQ ID NO: 12, the serum IgG of the dengue patients against the amino acid sequence of SEQ ID NO: 14, the serum IgG of the dengue patients against the amino acid sequence of SEQ ID NO: 15, the serum IgM of the dengue patients against the amino acid sequence of SEQ ID NO: 2, or any combination thereof is used to distinguish the dengue patients with mild case from the hospitalized severe case.

In one embodiment, the serum IgM of the dengue patients against the amino acid sequence of SEQ ID NO: 2, the serum IgM of the dengue patients against the amino acid sequence of SEQ ID NO: 4, the serum IgM of the dengue patients against the amino acid sequence of SEQ ID NO: 5, or any combination thereof is used to distinguish the dengue patients with mild case from the hospitalized severe case.

The present disclosure further provides a use of a flavivirus protein microarray for distinguishing dengue patients with mild case and hospitalized non-severe case from hospitalized severe case. The flavivirus protein microarray comprises:
a substrate including a plurality of protein array blocks on a surface of the substrate; and
at least one protein immobilized on each of the plurality of protein array blocks, wherein the at least one protein comprises an amino acid sequence of SEQ ID NO: 3 and an amino acid sequence of SEQ ID NO: 4, and
wherein a combination of a serum IgG of the dengue patients against the amino acid sequence of SEQ ID NO: 3 and the serum IgG of the dengue patients against the amino acid sequence of SEQ ID NO: 4 is used to distinguish the dengue patients with the mild case and the hospitalized non-severe case from the hospitalized severe case.

In one embodiment, the at least one protein further comprises an amino acid sequence of SEQ ID NO: 8, and wherein a combination of the serum IgG of the dengue patients against the amino acid sequence of SEQ ID NO: 3, the serum IgG of the dengue patients against the amino acid sequence of SEQ ID NO: 4, and the serum IgM of the dengue patients against the amino acid sequence of SEQ ID NO: 8 is used to distinguish the dengue patients with the mild case and the hospitalized non-severe case from the hospitalized severe case.

The present disclosure further provides a method for distinguishing dengue patients with mild case from hospitalized non-severe case and hospitalized severe case. The method comprises the steps of:
providing a flavivirus protein microarray of claim 1;
adding a non-protein blocking reagent to the plurality of protein array blocks of the flavivirus protein microarray for reaction to obtain a first flavivirus protein microarray;
providing a serum from one of the dengue patients with mild case, hospitalized non-severe case or hospitalized severe case, adding the serum to the first protein microarray for reaction, and then washing to obtain a second flavivirus protein microarray;
providing a fluorescently labeled anti-human immunoglobulin antibody and adding the fluorescently labeled anti-human immunoglobulin antibody to the second flavivirus protein microarray, and reacting for 50 to 70 minutes followed by washing to obtain a third flavivirus protein microarray; and
reading an optical signal generated from the third flavivirus protein microarray by a signal reader to quantify the fluorescently labeled anti-human immunoglobulin antibody.

In one embodiment, the fluorescently labeled anti-human immunoglobulin antibody recognizes a human immunoglobulin G (IgG), a human immunoglobulin M (IgM), or a combination thereof.

In one embodiment, a fluorescence used for the fluorescently labeled anti-human immunoglobulin antibody comprises cyanine dye Cy3 or cyanine dye Cy5.

The flavivirus protein microarray of the present disclosure may detect picograms of antibody against flavivirus protein. Therefore, the sensitivity of the flavivirus protein microarray of the present disclosure is much higher than commercial ELISA or rapid test. Moreover, the flavivirus protein microarray of the present disclosure may accurately distinguish dengue patients with mild case (group A) from hospitalized non-severe case (group B), distinguish dengue patients with mild case (group A) from hospitalized severe case (group C), and distinguish dengue patients with mild case (group A) from hospitalized non-severe and ICU cases (groups BC) in 2.5 hours and only 0.25 μL of patient serum is needed for examination. Therefore, the present disclosure provides the flavivirus protein microarray with low cost and high sensitivity may be used to efficiently predict that whether a dengue patient with mild case will develop into hospitalized non-severe case or hospitalized severe case, thereby reducing mortality.

### BRIEF DESCRIPTION OF DRAWINGS

FIG. 1 shows the design and result of quality control of a flavivirus protein microarray of the present application.

FIG. 2A, FIG. 2B, and FIG. 2C show the results of quality control of the flavivirus protein microarray of the present application. FIG. 2A indicates the result of quantification of anti-His signals. FIG. 2B indicates the result of repeatability of two anti-His experiments. FIG. 2C indicates the result of the sensitivity of the flavivirus protein microarray using an anti-DENV NS1 antibody. The anti-DENV NS1 antibody is serially diluted and then detects using Cy5-labeled anti-mouse. A calibration line and R2 value are calculated using linear regression. The absolute limit of detection (LOD) is determined by calculating the 3-fold standard deviation (SD) of the Y-intercept/slope of the calibration line.

FIG. 3A shows a process of sensitivity test of the flavivirus protein microarray. The Flavivirus protein microarray is first incubated with 200-fold diluted serum for an hour, washed, incubated with Cy3-labeled anti-human IgM and Cy5-labeled anti-human IgG for an hour, washed, dried, and scanned with a laser scanner.

FIG. 3B shows the result of serum IgG profiling with different dengue patient's severities by using the flavivirus protein microarray.

FIG. 3C shows the result of serum IgM profiling with different dengue patient's severities by using the flavivirus protein microarray.

FIGS. 4A-4H show the results of high-throughput detection of IgG responses against 4 dengue serotypes in healthy controls (H), mild (A), hospitalized non-severe (B), and severe (C) subjects. FIGS. 4A-4H show the analysis of IgG binding to different serotypes of dengue proteins in serum from healthy controls and dengue patients. One-Way ANOVA analyzed data with Tukey's multiple comparisons. ****p<0.0001, ***p<0.001, **p<0.01, *p<0.05 compared to the mean of each group with the mean of each column.

FIGS. 5A-5I show the results of high-throughput detection of IgG responses against 4 dengue serotypes in healthy controls (H), mild (A), hospitalized non-severe (B), and severe (C) subjects. FIGS. 5A-5I show the analysis of IgG binding to different flavivirus proteins in serum from healthy controls and dengue patients. One-Way ANOVA analyzed data with Tukey's multiple comparisons. ****p<0.0001, ***p<0.001, **p<0.01, *p<0.05 compared to the mean of each group with the mean of each column.

FIGS. 6A-6H show the results of high-throughput detection of IgM responses against 4 dengue serotypes in healthy controls (H), mild (A), hospitalized non-severe (B), and severe (C) subjects. FIGS. 6A-6H show the analysis of IgM binding to different serotypes of dengue proteins in serum from healthy controls and dengue patients. One-Way ANOVA analyzed data with Tukey's multiple comparisons. ****p<0.0001, ***p<0.001, **p<0.01, *p<0.05 compared to the mean of each group with the mean of each column.

FIGS. 7A-7I show the results of high-throughput detection of IgM responses against 4 dengue serotypes in healthy controls (H), mild (A), hospitalized non-severe (B), and severe (C) subjects. FIGS. 5A-5I show the analysis of IgM binding to different flavivirus proteins in serum from healthy controls and dengue patients. One-Way ANOVA analyzed data with Tukey's multiple comparisons. ****p<0.0001, ***p<0.001, **p<0.01, *p<0.05 compared to the mean of each group with the mean of each column.

FIGS. 8A-8H show the results of selection of the best combination markers from serum profiling to distinguish dengue patients with different severities. Markers shown in FIGS. 8A-8H are selected by logistic regression that combined the best markers and presented using ROC curves.

FIGS. 9A-9C show the results of selection of 1-3 markers, including single marker, tow markers in combine, and three markers in combine, from serum profiling to distinguish dengue patients with different severities. Markers shown in FIGS. 9A-9C are selected by logistic regression that combined one to three individual markers and presented using ROC curves.

FIGS. 10A-10B show the results of selection of 1-3 markers, including single marker, tow markers in combine, and three markers in combine, from serum profiling to distinguish distinguishing dengue patients with mild case from hospitalized non-severe and ICU dengue patients. Markers shown in FIGS. 10A-10B are selected by logistic regression that combined one to three individual markers and presented using ROC curves.

### DETAILED DESCRIPTION OF PREFERRED EMBODIMENTS

The following provides specific embodiments to illustrate the implementation of the present disclosure. A person having ordinary skill in the art can understand other advantages and effects of the present disclosure from the contents disclosed in the present specification. However, the exemplary embodiments disclosed in the present disclosure are only for illustrative purposes and should not be regarded as limiting the scope of the present disclosure. In other words, the present disclosure can also be implemented or applied by other different specific embodiments, and various details in the present specification can also be modified and changes based on different viewpoints and applications without departing from the concept of the present disclosure.

Unless otherwise indicated herein, the singular forms “one” and “the” used in the specification and the appended claims of the present disclosure include the plural.

Unless otherwise indicated herein, the term “or” used in the specification and the appended claims of the present disclosure includes the meaning of “and/or”.

Unless otherwise indicated herein, the term “hospitalized case” used in the specification and the appended claims of the present disclosure includes the meaning of “hospitalized non-severe case” and “hospitalized severe case”.

### Preparation Example 1: Preparation of a Flavivirus Protein Microarray

Seventeen flavivirus proteins with histidine tag (His-tag) listed in Table 1 are purchased from Native Antigen company, and each of the proteins is used as a biomarker for detecting virus infection. The flavivirus proteins comprise envelope protein (E protein) from type I dengue virus (hereinafter referred to as “DENV1-E” and abbreviated as “IE”) having an amino acid sequence of SEQ ID NO: 1, NS1 protein from type I dengue virus (hereinafter referred to as “DENV1-NS1” and abbreviated as “INS1”) having an amino acid sequence of SEQ ID NO: 2, E protein from type II dengue virus (hereinafter referred to as “DENV2-E” and abbreviated as “IIE”) having an amino acid sequence of SEQ ID NO: 3, NS1 protein from type II dengue virus (hereinafter referred to as “DENV2-NS1” and abbreviated as “IINS1”) having an amino acid sequence of SEQ ID NO: 4, E protein from type III dengue virus (hereinafter referred to as “DENV3-E” and abbreviated as “IIIE”) having an amino acid sequence of SEQ ID NO: 5, NS1 protein from type III dengue virus (hereinafter referred to as “DENV3-NS1” and abbreviated as “IIINS1”) having an amino acid sequence of SEQ ID NO: 6, E protein from type IV dengue virus (hereinafter referred to as “DENV4-E” and abbreviated as “IVE”) having an amino acid sequence of SEQ ID NO: 7, NS1 protein from type IV dengue virus (hereinafter referred to as “DENV4-NS1” and abbreviated as “IVNS1”) having an amino acid sequence of SEQ ID NO: 8, NS1 protein from Zika virus (hereinafter referred to as “ZIKA-NS1” and abbreviated as “ZVNS1”) having an amino acid sequence of SEQ ID NO: 9, E protein from Zika virus (Suriname strain) (hereinafter referred to as “ZIKASU-E” and abbreviated as “ZSE”) having an amino acid sequence of SEQ ID NO: 10, NS1 protein from Zika virus (Suriname strain) (hereinafter referred to as “ZIKASU-NS1” and abbreviated as “ZSNS1”) having an amino acid sequence of SEQ ID NO: 11, E protein from West Nile virus (hereinafter referred to as “WNV-E” and abbreviated as “WE”) having an amino acid sequence of SEQ ID NO: 12, NS1 protein from West Nile virus (hereinafter referred to as “WNV-NS1” and abbreviated as “WNS1”) having an amino acid sequence of SEQ ID NO: 13, E protein from Japanese encephalitis virus (hereinafter referred to as “JEV-E” and abbreviated as “JE”) having an amino acid sequence of SEQ ID NO: 14, NS1 protein from Japanese encephalitis virus (hereinafter referred to as “JEV-NS1” and abbreviated as “JNS1”) having an amino acid sequence of SEQ ID NO: 15, NS1 protein from Tick-borne Encephalitis virus (hereinafter referred to as “TEBV-NS1” and abbreviated as “TNS1”) having an amino acid sequence of SEQ ID NO: 16, and NS1 protein from Yellow fever virus (hereinafter referred to as “YFV-NS1” and abbreviated as “YNS1”) having an amino acid sequence of SEQ ID NO: 17.

TABLE 1
List of seventeen flavivirus proteins.

| Virus | Protein | Amino acid |
|---|---|---|
| Type I dengue virus | DENV1-E | SEQ ID NO: 1 |
| | DENV1-NS1 | SEQ ID NO: 2 |
| Type II dengue virus | DENV2-E | SEQ ID NO: 3 |
| | DENV2-NS1 | SEQ ID NO: 4 |
| Type III dengue virus | DENV3-E | SEQ ID NO: 5 |
| | DENV3-NS1 | SEQ ID NO: 6 |
| Type IV dengue virus | DENV4-E | SEQ ID NO: 7 |
| | DENV4-NS1 | SEQ ID NO: 8 |
| Zika virus | ZVNS1 | SEQ ID NO: 9 |
| Zika virus (Suriname strain) | ZIKASU-E | SEQ ID NO: 10 |
| | ZIKASU-NS1 | SEQ ID NO: 11 |
| West Nile virus | WNV-E | SEQ ID NO: 12 |
| | WNV-NS1 | SEQ ID NO: 13 |
| Japanese encephalitis virus | JEV-E | SEQ ID NO: 14 |
| | JEV-NS1 | SEQ ID NO: 15 |
| Tick-borne Encephalitis virus | TEV-NS1 | SEQ ID NO: 16 |
| Yellow fever virus | YFV-NS1 | SEQ ID NO: 17 |

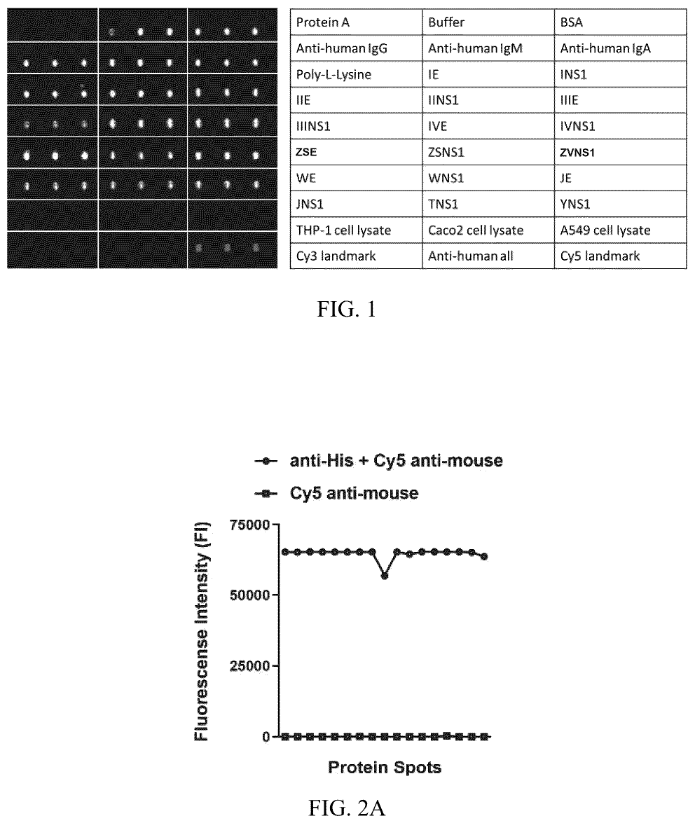

Referring to FIG. 1, twenty-eight samples, including seventeen flavivirus proteins, cell lysate, and control samples shown in Table 2 are prepared for printing. Proteins are prepared at 0.1 mg/ml in PBS with 30% glycerol. The cell lysate is prepared by harvesting 4×10⁶ cells, washed, and extracted by commercial kit (ThermoFisher, no. 89842). The twenty-eight samples are printed on the slides in triplicate in 14 identical blocks by using a microarray contact spotting system (CapitalBio SmartArrayer™ 136, Mainland China) to obtain a flavivirus protein microarray. After printing, the flavivirus protein microarray is immobilized for at least 8 hours, vacuum-sealed, and stored at −80° C.

TABLE 2
List of cell lysate and control samples.

| Cell lysate, Control Group | |
|---|---|
| and Experimental Group | Chemical Compounds |
| Cell lysate | A549 lysis |
| | THP1 lysis |
| | CaCo2 lysis |
| Negative control group | Bovine serum albumin |
| | TBST buffer (Tris buffer and 0.1% Tween |
| | 20) |
| Positive control group | Cy3-landmark |
| | Cy5- landmark |
| Experimental group | Poly-L-lysine |
| | Protein A |
| | Anti-human-IgG |
| | Anti-human-IgA |
| | Anti-human-IgM |

### Example 1: Examination of Quality and Sensitivity of the Flavivirus Protein Microarray

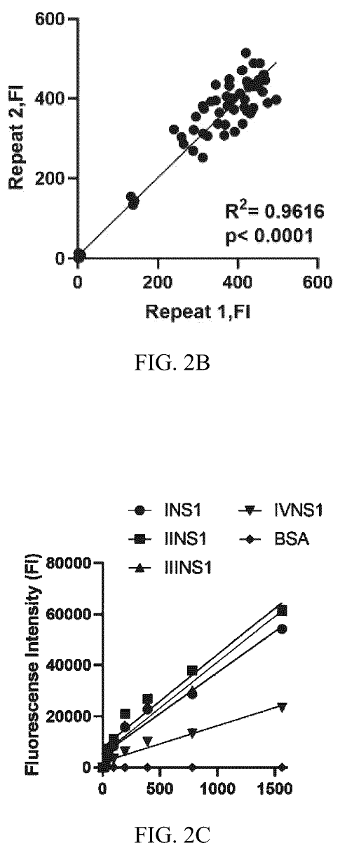

To confirm whether the His-tagged proteins are immobilized on the slide surface, the flavivirus protein microarray is stained with anti-His mouse monoclonal antibody followed by a Cy5-labeled anti-mouse IgG and then dried for scanning Cy3 and Cy5 signals with power 25% and 30% (Caduceus Biotechnology, #SpinScan) after washing. Referring to FIG. 1 and FIG. 2A, compared to the secondary antibody, the anti-His showed significantly higher signals. Referring to FIG. 2B, the reproducibility of this anti-His assay may be repeated in another assay with an R-square equal to 0.9616.

Moreover, to confirm the sensitivity of the flavivirus protein microarray, a pan anti-DENV NS1 antibody (LEADGENE, no. 12111) is used to detect multiple NS1 proteins on the flavivirus protein microarray. Referring to FIG. 2C, the pan anti-DENV NS1 antibody recognized the multiple NS1 proteins, predominately recognized dengue viruses. The LOD of pan anti-DENV NS1 for INS1=183.26 pg, IINS1=214.05 pg, IIINS1=172.86 pg, and IVNS1=172.23 pg. The result shows that the LOD from the flavivirus protein microarray is more sensitive than commercial ELISA or rapid test.

### Example 2: Investigation of IgG and IgM Profiles in Subjects with Different Dengue Patients' Severities

(1) For Profiling the IgG in Subjects with Different Dengue Patients' Severities:

To investigate the relationship between humoral immune responses and dengue outcomes, 86 dengue patients infected by type II dengue virus with different severity and 36 healthy controls are recruited for serum collection and the serum are analyzed by using the flavivirus protein microarray. According to the guideline to classification recommended by WHO in 2009, among 86 dengue patients, 21 patients are classified as mild (group A), 29 patients are classified as hospitalized non-severe cases (group B), and 36 patients are classified as hospitalized severe cases (group C). The day the patient first developed a fever is considered day 0 of illness and only acute patients within seven days of illness are included. The baseline characteristics of 86 dengue patients are shown in Table 3.

TABLE 3
Baseline characteristics of dengue patients.

| | WHO Classification | | | | | |
|---|---|---|---|---|---|---|
| | Group A | | Group B | | Group C | |
| | (n = 21) | | (n = 29) | | (n = 36) | |
| Age (years), (mean (SD)) | 43 | (20) | 64 | (20) | 68 | (16) |
| Gender, male (%) | 10 | (48%) | 11 | (38%) | 24 | (67%) |
| Hospital days | 0 | | 9 | (6) | 21 | (16) |
| ICU days | 0 | | 0 | | 7 | (11) |
| Inspection day, (mean (SD)) | 2 | (2) | 4 | (2) | 4 | (2) |

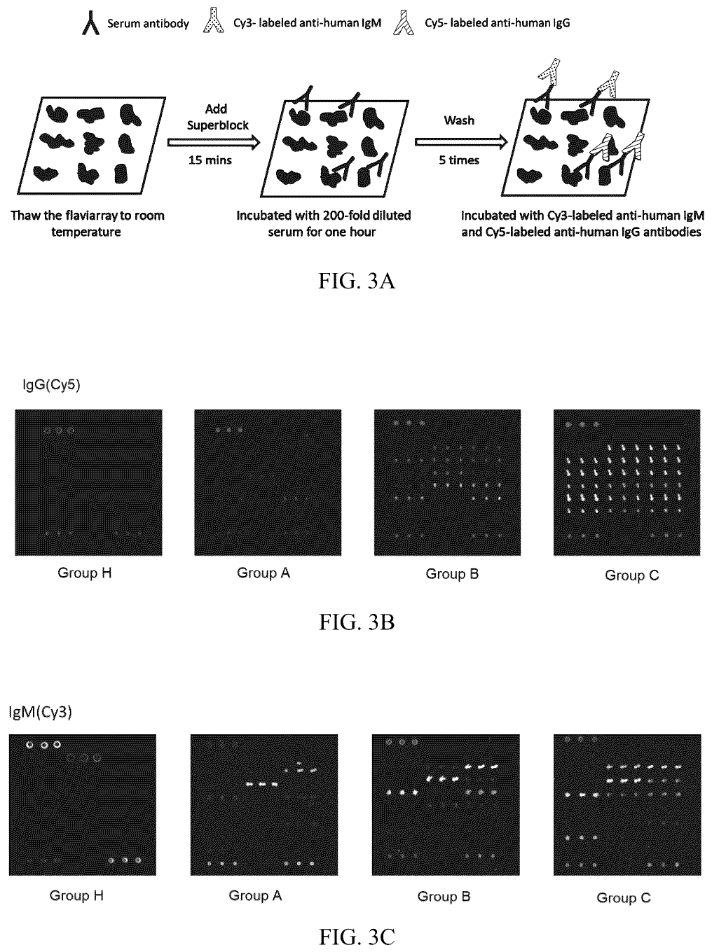

Referring to FIG. 3A, the cross-reactivities to various dengue serotypes and other Flaviviruses in subjects with different dengue severities are examined. The experimental process takes about 2.5 hours, and only 0.25 μL of patient serum is needed for each block. After blocking, the flavivirus protein microarray is incubated with serum for an hour, washed, and visualized with Cy3-labeled anti-human IgM and Cy5-labeled anti-human IgG.

To analyze the antibody response of dengue patients with different severity, serum from subjects with mild, hospitalized non-severe, severe, and healthy participants are collected. For the dengue patients, the average data are collected after the fever is 2±2 days in group A, 4±2 days in group B, and 4±2 days in group C. Referring to FIG. 3B and FIG. 3C, the results show a positive correlation between cross-reactivity between Flaviviruses and disease severity.

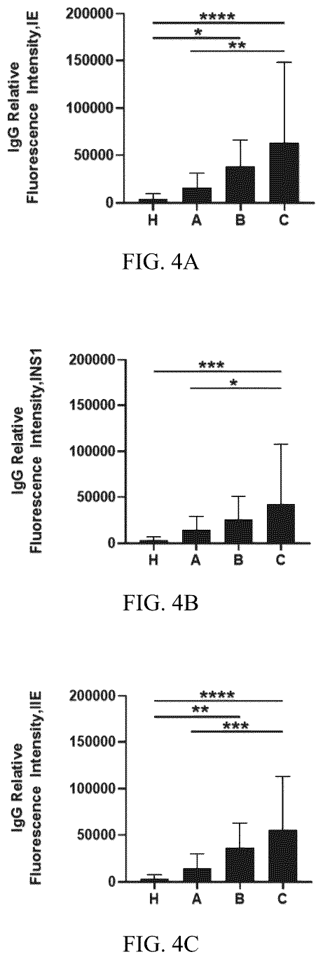

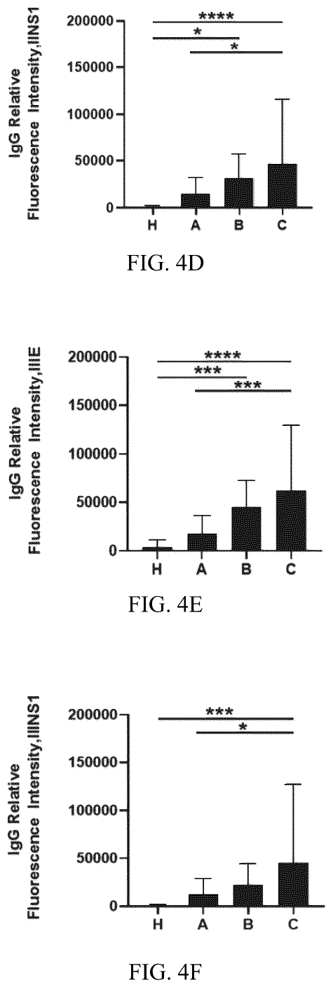

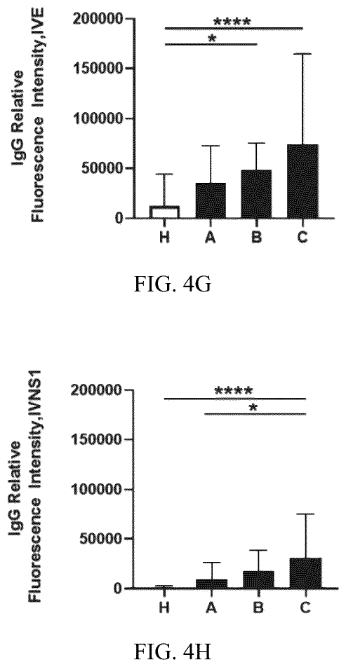

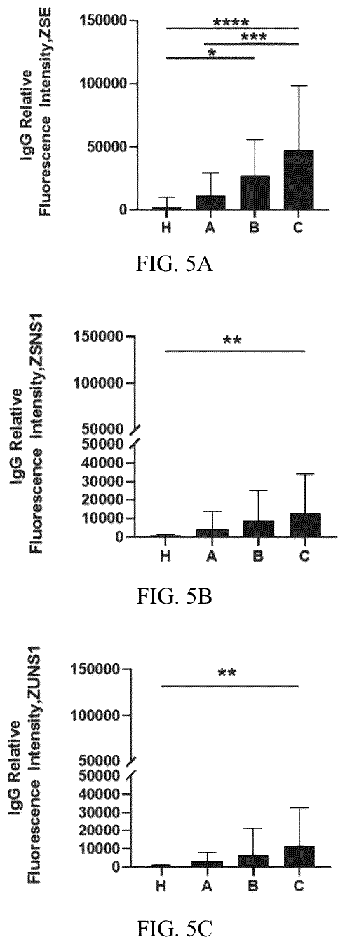

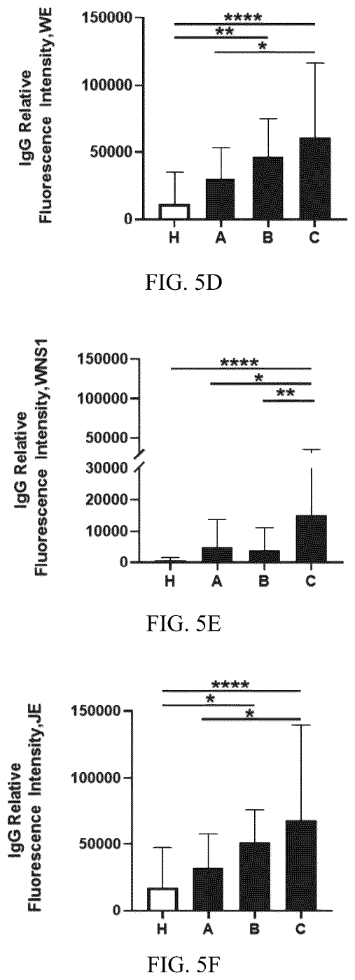

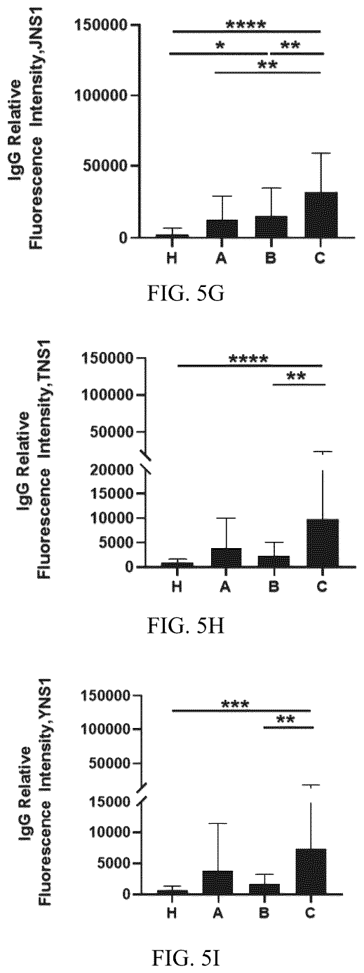

Moreover, referring to FIGS. 4A-4H and FIGS. 5A-5I, after analyzing the sera of dengue patients with different severities using the flavivirus protein microarray, the results show that the sera of severe patients have higher signals for dengue proteins and flavivirus proteins. That is, the severe patients infected by type II dengue virus, especially dengue patients in group C, have higher immune responses and higher cross-reactivities to type I dengue virus, type III dengue virus, type IV dengue virus and other flaviviruses. In addition, except for the type IV dengue E protein, the serum IgG signals of severe patients to the four types of dengue proteins were significantly higher than those of mild patients (FIGS. 4A-4H), which can distinguish between mild and hospitalized severe cases.

(2) For Profiling the IgM in Subjects with Different Dengue Patients' Severities:

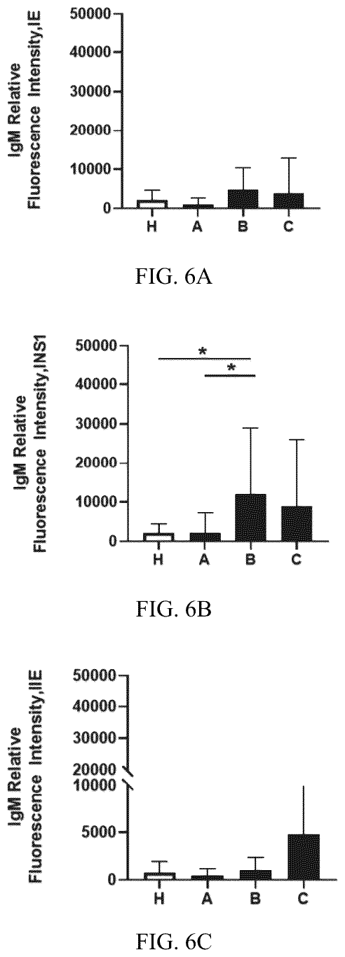

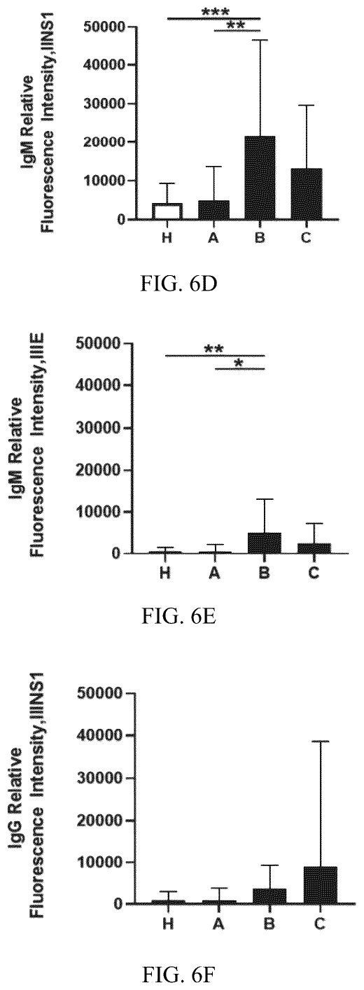

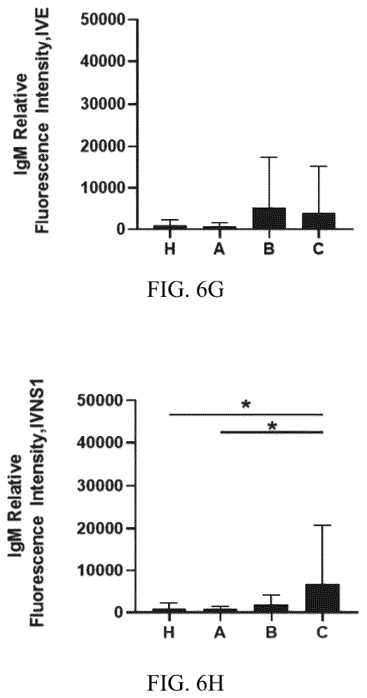

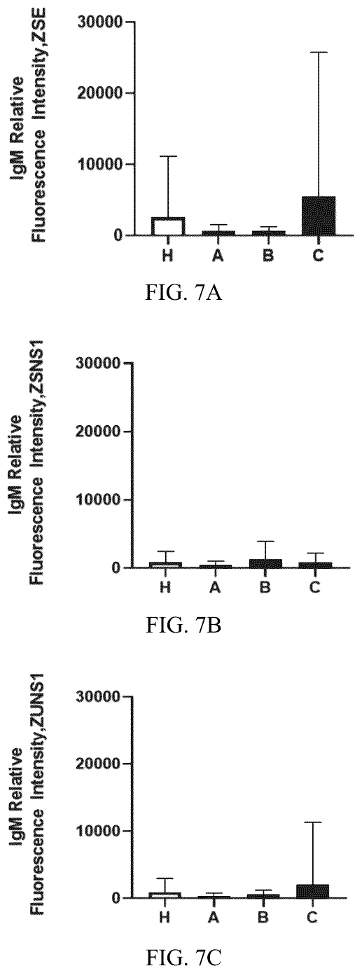

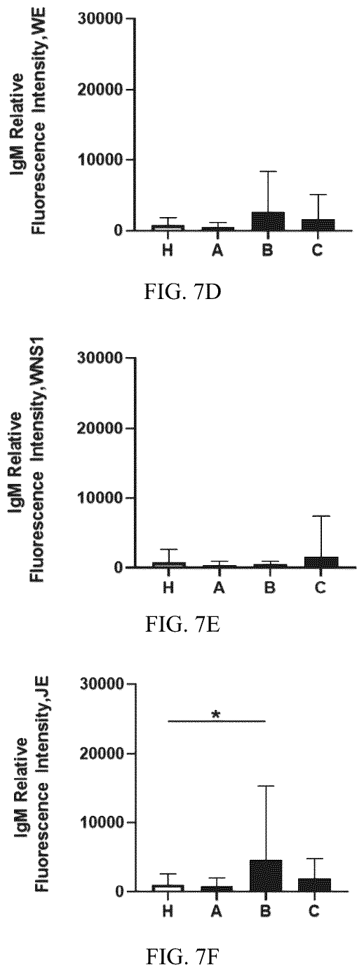

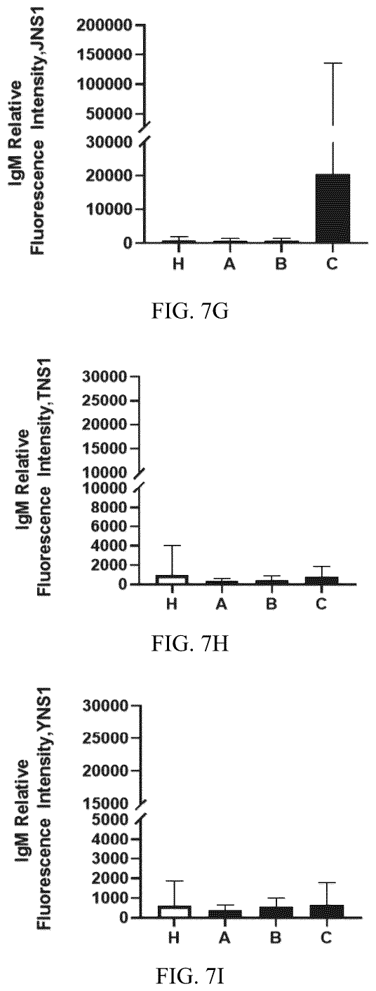

Since IgM generally has high specificity and appears in patients' serum in the early stage of infections, the serum IgG and IgM are labeled in one test to compare the immune responses as well as the specificities. Referring FIGS. 6A-6H and FIGS. 7A-7I, the results show that the cross-reactivity of IgM is lower compared with IgG. Moreover, the signal intensities of IgM against various viral antigens are lower than those of IgG. Furthermore, the signal intensity of IgM does not increase with the severity of the disease like IgG. However, among the IgM against INS1, IINS1, IIIE, and IVE, the signal value of hospitalized non-severe patients is higher than that of severe patients. In addition, in the three proteins of INS1, IINS1, and IIIE, it is possible to tell the difference between mild and severe patients. Therefore, the three proteins of INS1, IINS1, and IIIE may be used to decide if a subject should be hospitalized.

### Example 3: Selection of Markers for Diagnosis or Severity Prediction

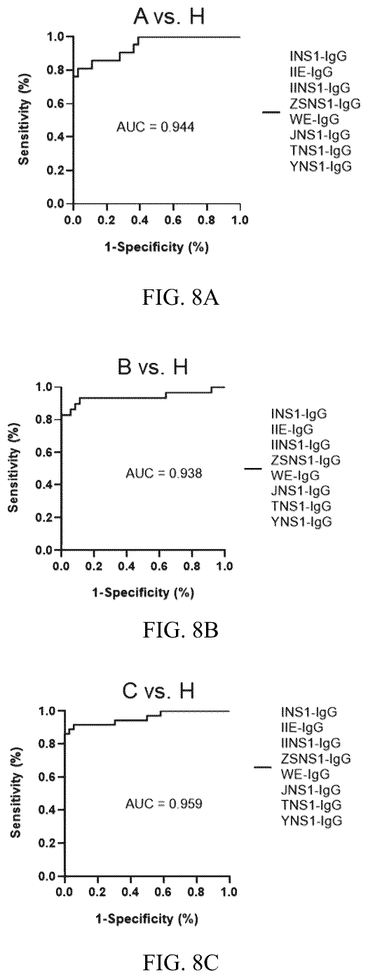

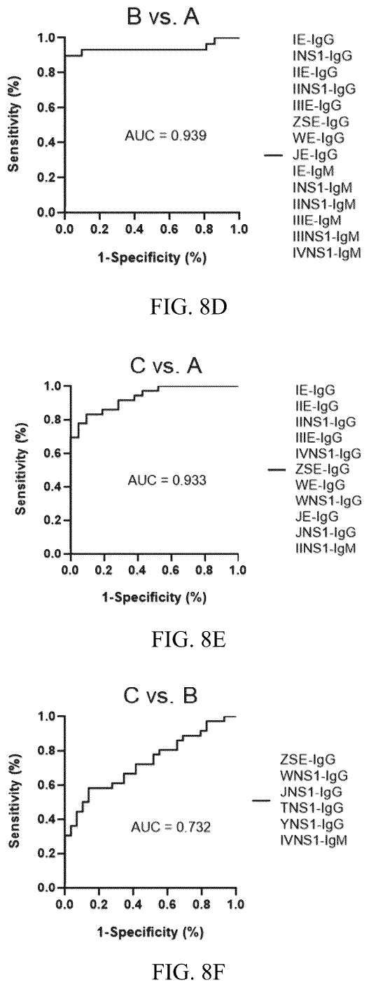

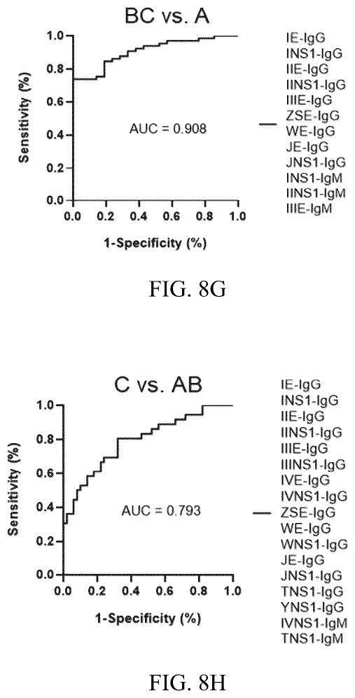

To select markers for diagnosis or severity prediction purposes, the logistic regression that combines the best markers and presents using receiver operating characteristic (ROC) curves is performed. Referring to FIG. 8A to FIG. 8H, the performance of the best markers is quantified via area under curves (AUCs), 0.944 for A vs H comparison (FIG. 8A), 0.938 for B vs H comparison (FIG. 8B), 0.959 for C vs H comparison (FIG. 8C), 0.939 for B vs A comparison (FIG. 8D), 0.933 for C vs A comparison (FIG. 8E), 0.732 for C vs B comparison (FIG. 8F), 0.908 for BC vs A comparison (FIG. 8G), and 0.793 for C vs AB comparison (FIG. 8H).

The results show that the markers shown in FIG. 8A to FIG. 8C may provide high performance for distinguishing dengue patients from healthy controls cases (AUC>0.9). Moreover, the markers shown in FIG. 8D, FIG. 8E, FIG. 8G may provide high performance for distinguishing dengue patients with mild case (group A) from hospitalized non-severe case (group B), distinguishing dengue patients with mild case (group A) from hospitalized severe cases (group C), and distinguishing dengue patients with mild case (group A) from hospitalized non-severe and ICU cases (groups BC) (AUC>0.9).

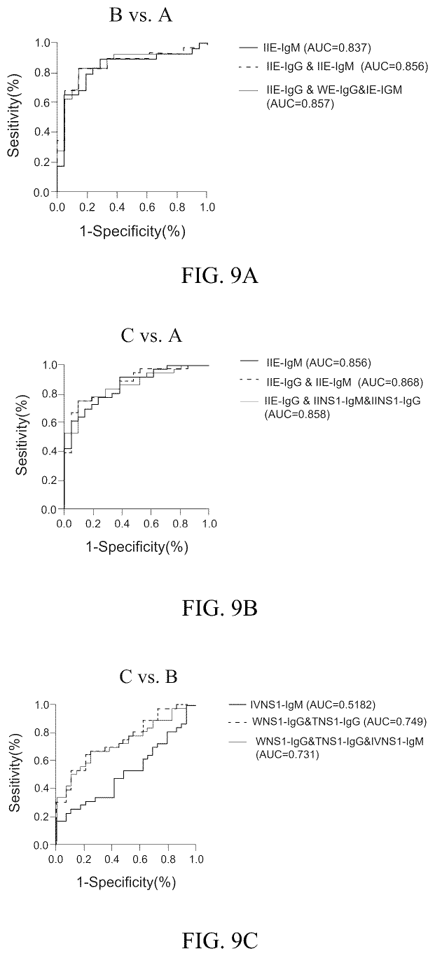

To increase the flexibility for the point of care applications such as ELISA or rapid test, one to three markers are selected. Referring to FIG. 9A to FIG. 9C, severity markers including single marker to triple markers are discovered by comparing group A, group B, and group C. The results show that in B vs A comparison, the best performed markers are single IE-IgM (AUC=0.837), double IIE-IgG+IE-IgM (AUC=0.856), and triple IIE-IgG+WE-IgG+IE-IgM (AUC=0.857) (FIG. 9A). In C vs A comparison, the best performed markers are single IIE-IgG (AUC=0.856), double IIE-IgG+IINS1-IgM (AUC=0.868), and triple IIE-IgG+IINS1-IgM+IINS1-IgG (AUC=0.858) (FIG. 9B). In C vs B comparison, the best performed markers are single WNS1-IgG (AUC=0.74), double WNS1-IgG+TNS1-IgG (AUC=0.749), and triple WNS1-IgG+TNS1-IgG+IVNS1 (AUC=0.731) (FIG. 9C).

Therefore, the markers shown in FIG. 9A and FIG. 9B may provide high performance for distinguishing dengue patients with mild case (group A) from hospitalized non-severe case (group B) and distinguishing dengue patients with mild case (group A) from hospitalized severe cases (group C) (AUC>0.8).

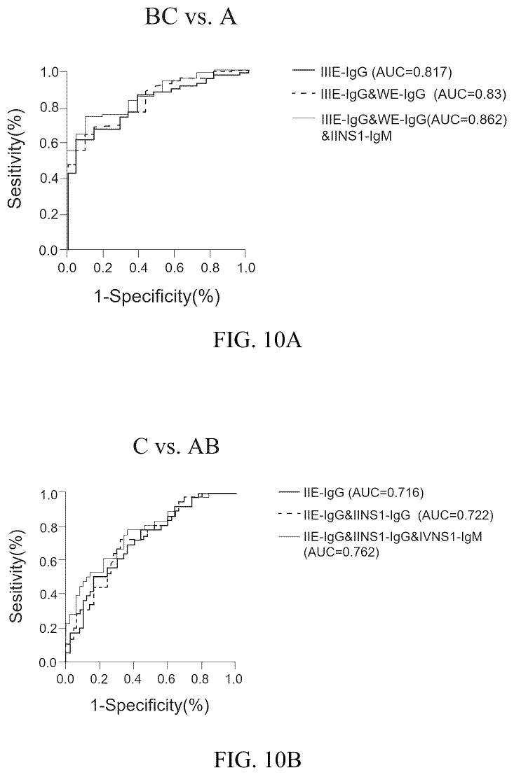

Moreover, referring to FIG. 10A and FIG. 10B, hospitalized markers and severe markers including single marker to triple markers are discovered by comparing groups BC vs group A and group C vs groups AB for distinguishing hospitalized non-severe and ICU patients. The results show that in BC vs A comparison, the best performed markers are single IIIE-IgG (AUC=0.817), double IIIE-IgG+WE-IgG (AUC-0.830), and triple IIIE-IgG+WE-IgG+IINS1-IgM (AUC=0.862) (FIG. 10A). In C vs AB comparison, the best performed markers are single IIE-IgG (AUC=0.716), double IIE-IgG+IINS1-IgG (AUC=0.722), and triple IIE-IgG+IINS1-IgG+IVNS1-IgM (AUC=0.762) (FIG. 10B).

Therefore, the markers shown in FIG. 10A and FIG. 10B may provide high performance for distinguishing dengue patients with mild case (group A) from hospitalized non-severe and ICU cases (groups BC) (AUC>0.8).

Based on the results described above, the flavivirus protein microarray of the present disclosure may detect picograms of antibody against flavivirus protein. Therefore, the sensitivity of the flavivirus protein microarray of the present disclosure is much higher than commercial ELISA or rapid test. Moreover, the flavivirus protein microarray of the present disclosure may accurately distinguish dengue patients with mild case (group A) from hospitalized non-severe case (group B), distinguish dengue patients with mild case (group A) from hospitalized severe case (group C), and distinguish dengue patients with mild case (group A) from hospitalized non-severe and ICU cases (groups BC) in 2.5 hours and only 0.25 μL of patient serum is needed for examination. Therefore, the present disclosure provides the flavivirus protein microarray with low cost and high sensitivity a may be used to efficiently predict that whether a dengue patient with mild case will develop into hospitalized non-severe case or hospitalized severe case, thereby reducing mortality.

Although the present disclosure has been disclosed in preferred embodiments, it is not intended to limit the present disclosure. A person having ordinary skill in the art can make various changes and modifications without departing from the concept and scope of the present disclosure. Therefore, the claimed scope of the present disclosure shall be based on the scope defined by the attached claims of the patent disclosure.

What is claimed is:

1. A use of a flavivirus protein microarray for distinguishing dengue patients with mild case from hospitalized case, wherein the hospitalized case comprises hospitalized non-severe case and hospitalized severe case; the flavivirus protein microarray comprises:
a substrate including a plurality of protein array blocks on a surface of the substrate; and
at least one protein immobilized on each of the plurality of protein array blocks, wherein the at least one protein comprises an amino acid sequence of SEQ ID NO: 1, an amino acid sequence of SEQ ID NO: 3, and an amino acid sequence of SEQ ID NO: 13, and
wherein a serum IgM of the dengue patients against the amino acid sequence of SEQ ID NO: 1 is used to distinguish the dengue patients with mild case from the hospitalized non-severe case, a serum IgG of the dengue patients against the amino acid sequence of SEQ ID NO: 3 is used to distinguish the dengue patients with mild case from the hospitalized severe case, and the serum IgG of the dengue patients against the amino acid sequence of SEQ ID NO: 13 is used to distinguish the dengue patients with mild case from the hospitalized case.

2. The use according to claim 1, wherein a combination of the serum IgM of the dengue patients against the amino acid sequence of SEQ ID NO: 1 and the serum IgG of the dengue patients against the amino acid sequence of SEQ ID NO: 3 is used to distinguish the dengue patients with mild case from the hospitalized non-severe case.

3. The use according to claim 1, wherein the at least one protein further comprises an amino acid sequence of SEQ ID NO: 4, and wherein a combination of the serum IgG of the dengue patients against the amino acid sequence of SEQ ID NO: 3 and the serum IgM of the dengue patients against the amino acid sequence of SEQ ID NO: 4 is used to distinguish the dengue patients with mild case from the hospitalized severe case.

4. The use according to claim 1, wherein the at least one protein further comprises an amino acid sequence of SEQ ID NO: 16, and wherein a combination of the serum IgG of the dengue patients against the amino acid sequence of SEQ ID NO: 13 and the serum IgG of the dengue patients against the amino acid sequence of SEQ ID NO: 16 is used to distinguish the dengue patients with mild case from the hospitalized case.

5. The use according to claim 2, wherein the at least one protein further comprises an amino acid sequence of SEQ ID NO: 12, and wherein a combination of the serum IgM of the dengue patients against the amino acid sequence of SEQ ID NO: 1, the serum IgG of the dengue patients against the amino acid sequence of SEQ ID NO: 3, and the serum IgG of the dengue patients against the amino acid sequence of SEQ ID NO: 12 is used to distinguish the dengue patients with mild case from the hospitalized non-severe case.

6. The use according to claim 3, wherein a combination of the serum IgG of the dengue patients against the amino acid sequence of SEQ ID NO: 3, the serum IgM of the dengue patients against the amino acid sequence of SEQ ID NO: 4, and the serum IgG of the dengue patients against the amino acid sequence of SEQ ID NO: 4 is used to distinguish the dengue patients with mild case from the hospitalized severe case.

7. The use according to claim 4, wherein the at least one protein further comprises an amino acid sequence of SEQ ID NO: 8, a combination of the serum IgG of the dengue patients against the amino acid sequence of SEQ ID NO: 13, the serum IgG of the dengue patients against the amino acid sequence of SEQ ID NO: 16, and the serum IgM of the dengue patients against the amino acid sequence of SEQ ID NO: 8 is used to distinguish the dengue patients with mild case from the hospitalized case.

8. The use according to claim 1, wherein the at least one protein further comprises an amino acid sequence of SEQ ID NO: 5 and an amino acid sequence of SEQ ID NO: 12, and wherein a combination of the serum IgG of the dengue patients against the amino acid sequence of SEQ ID NO: 5 and the serum IgG of the dengue patients against the amino acid sequence of SEQ ID NO: 12 is used to distinguish the dengue patients with mild case from the hospitalized case.

9. The use according to claim 8, wherein the at least one protein further comprises an amino acid sequence of SEQ ID NO: 4; and wherein a combination of the serum IgG of the dengue patients against the amino acid sequence of SEQ ID NO: 5, the serum IgG of the dengue patients against the amino acid sequence of SEQ ID NO: 12, and the serum IgM of the dengue patients against the amino acid sequence of SEQ ID NO: 4 is used to distinguish the dengue patients with mild case from the hospitalized case.

10. The use according to claim 1, wherein the at least one protein further comprises an amino acid sequence of SEQ ID NO: 2, an amino acid sequence of SEQ ID NO: 4, an amino acid sequence of SEQ ID NO: 5, an amino acid sequence of SEQ ID NO: 10, an amino acid sequence of SEQ ID NO: 12, an amino acid sequence of SEQ ID NO: 14, or any combination thereof, and wherein the serum IgG of the dengue patients against the amino acid sequence of SEQ ID NO: 1, serum IgG of the dengue patients against the amino acid sequence of SEQ ID NO: 2, the serum IgG of the dengue patients against the amino acid sequence of SEQ ID NO: 4, the serum IgG of the amino acid sequence of SEQ ID NO: 5, the serum IgG of the dengue patients against the amino acid sequence of SEQ ID NO: 10, the serum IgG of the dengue patients against the amino acid sequence of SEQ ID NO: 12, the serum IgG of the dengue patients against the amino acid sequence of SEQ ID NO: 14, or any combination thereof is used to distinguish the dengue patients with mild case from the hospitalized non-severe case.

11. The use according to claim 10, wherein the at least one protein further comprises an amino acid sequence of SEQ ID NO: 6, and wherein the serum IgM of the amino acid sequence of SEQ ID NO: 2, the serum IgM of the dengue patients against the amino acid sequence of SEQ ID NO: 4, the serum IgM of the amino acid sequence of SEQ ID NO: 5, the serum IgM of the amino acid sequence of SEQ ID NO: 6, or any combination thereof is used to distinguish the dengue patients with mild case from the hospitalized non-severe case.

12. The use according to claim 1, wherein the at least one protein further comprises an amino acid sequence of SEQ ID NO: 4, an amino acid sequence of SEQ ID NO: 5, an amino acid sequence of SEQ ID NO: 8, an amino acid sequence of SEQ ID NO: 10, an amino acid sequence of SEQ ID NO: 12, an amino acid sequence of SEQ ID NO: 14, an amino acid sequence of SEQ ID NO: 15, or any combination thereof, and wherein the serum IgG of the dengue patients against the amino acid sequence of SEQ ID NO: 1, the serum IgG of the dengue patients against the amino acid sequence of SEQ ID NO: 4, the serum IgG of the dengue patients against the amino acid sequence of SEQ ID NO: 5, the serum IgG of the dengue patients against the amino acid sequence of SEQ ID NO: 8, the serum IgG of the dengue patients against the amino acid sequence of SEQ ID NO: 10, the serum IgG of the dengue patients against the amino acid sequence of SEQ ID NO: 12, the serum IgG of the dengue patients against the amino acid sequence of SEQ ID NO: 14, the serum IgG of the dengue patients against the amino acid sequence of SEQ ID NO: 15, or any combination thereof is used to distinguish the dengue patients with mild case from the hospitalized severe case.

13. The use according to claim 12, wherein the serum IgM of the dengue patients against the amino acid sequence of SEQ ID NO: 4 is used to distinguish the dengue patients with mild case from the hospitalized severe case.

14. The use according to claim 1, wherein the at least one protein further comprises an amino acid sequence of SEQ ID NO: 2, an amino acid sequence of SEQ ID NO: 4, an amino acid sequence of SEQ ID NO: 5, an amino acid sequence of SEQ ID NO: 10, an amino acid sequence of SEQ ID NO: 12, an amino acid sequence of SEQ ID NO: 14, an amino acid sequence of SEQ ID NO: 15, or any combination thereof, and wherein the serum IgG of the dengue patients against the amino acid sequence of SEQ ID NO: 1, the serum IgG of the dengue patients against the amino acid sequence of SEQ ID NO: 2, the serum IgG of the dengue patients against the amino acid sequence of SEQ ID NO: 4, the serum IgG of the dengue patients against the amino acid sequence of SEQ ID NO: 5, the serum IgG of the dengue patients against the amino acid sequence of SEQ ID NO: 10, the serum IgG of the dengue patients against the amino acid sequence of SEQ ID NO: 12, the serum IgG of the dengue patients against the amino acid sequence of SEQ ID NO: 14, the serum IgG of the dengue patients against the amino acid sequence of SEQ ID NO: 15, or any combination thereof is used to distinguish the dengue patients with mild case from the hospitalized case.

15. The use according to claim 14, wherein the serum IgM of the dengue patients against the amino acid sequence of SEQ ID NO: 2, the serum IgM of the dengue patients against the amino acid sequence of SEQ ID NO: 4, the serum IgM of the dengue patients against the amino acid sequence of SEQ ID NO: 5, or any combination thereof is used to distinguish the dengue patients with mild case from the hospitalized case.

16. A use of a flavivirus protein microarray for distinguishing dengue patients with mild case and hospitalized non-severe case from hospitalized severe case, wherein the flavivirus protein microarray comprises:
a substrate including a plurality of protein array blocks on a surface of the substrate; and
at least one protein immobilized on each of the plurality of protein array blocks, wherein the at least one protein comprises an amino acid sequence of SEQ ID NO: 3 and an amino acid sequence of SEQ ID NO: 4, and
wherein a combination of a serum IgG of the dengue patients against the amino acid sequence of SEQ ID NO: 3 and the serum IgG of the dengue patients against the amino acid sequence of SEQ ID NO: 4 is used to distinguish the dengue patients with the mild case and the hospitalized non-severe case from the hospitalized severe case.

17. The use according to claim 13, wherein the at least one protein further comprises an amino acid sequence of SEQ ID NO: 8, and wherein a combination of the serum IgG of the dengue patients against the amino acid sequence of SEQ ID NO: 3, the serum IgG of the dengue patients against the amino acid sequence of SEQ ID NO: 4, and the serum IgM of the dengue patients against the amino acid sequence of SEQ ID NO: 8 is used to distinguish the dengue patients with the mild case and the hospitalized non-severe case from the hospitalized severe case.

18. A method for distinguishing dengue patients with mild case from hospitalized non-severe case and hospitalized severe case, comprising the steps of:
providing a flavivirus protein microarray of claim 1;
providing a serum from one of the dengue patients with mild case, hospitalized non-severe case or hospitalized severe case and adding the serum to the flavivirus protein microarray for reaction;
providing a fluorescently labeled anti-human immunoglobulin antibody and adding the fluorescently labeled anti-human immunoglobulin antibody to the flavivirus protein microarray for reaction; and
reading an optical signal generated from the flavivirus protein microarray by a signal reader to quantify the fluorescently labeled anti-human immunoglobulin antibody; and
distinguishing the dengue patients with mild case from the hospitalized non-severe case based upon the optical signal generated from the amino acid sequence of SEQ ID NO: 1, distinguishing the dengue patients with mild case from the hospitalized severe case based upon the optical signal generated from the amino acid sequence of SEQ ID NO: 3, and distinguishing the dengue patients with mild case from the hospitalized case based upon the optical signal generated from the amino acid sequence of SEQ ID NO: 13.

19. The method according to any one of claim 18, wherein the fluorescently labeled anti-human immunoglobulin antibody recognizes a human immunoglobulin G (IgG), a human immunoglobulin M (IgM), or a combination thereof.

20. The method according to any one of claim 18, wherein a fluorescence used for the fluorescently labeled anti-human immunoglobulin antibody comprises cyanine dye Cy3 or cyanine dye Cy5.


SEQUENCE LISTING (17 sequences)

SEQ ID NO: 1 - protein, 396 aa, Dengue virus
  source 1..396: note = Type I-envelope protein
MRCVGIGNRDFVEGLSGATWVDVVLEHGSCVTTMAKNKPTLDIELLKTEVTNPAVLRKLCIEAKISNTTTDSRCPTQGEATLVEEQDANFVCRRTVVDRGWGNGCGLFGKGSLLTCAKFKCVTKLEGKIVQYENLKYSVIVTVHTGDQHQVGNETTEHGTIATITPQAPTSEIQLTDYGTLTLDCSPRTGLDFNEVVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGVTQNGRLITANPIVTDKEKPINIETEPPFGESYIIVGAGEKALKLSWFKKGS

SEQ ID NO: 2 - protein, 352 aa, Dengue virus
  source 1..352: note = Type I-NS1 protein
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSA

SEQ ID NO: 3 - protein, 401 aa, Dengue virus
  source 1..401: note = Type II-envelope protein
MTMRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKIVQPENLEYTIVVTPHSGEEHAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIG

SEQ ID NO: 4 - protein, 352 aa, Dengue virus
  source 1..352: note = Type II-NS1 protein
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTA

SEQ ID NO: 5 - protein, 399 aa, Dengue virus
  source 1..399: note = Type III-Envelope protein
MTMRCVGVGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWASGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIG

SEQ ID NO: 6 - protein, 352 aa, Dengue virus
  source 1..352: note = Type III-NS1 protein
DMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSA

SEQ ID NO: 7 - protein, 399 aa, Dengue virus
  source 1..399: note = Type IV-Enveloope protein
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTAMITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIG

SEQ ID NO: 8 - protein, 352 aa, Dengue virus
  source 1..352: note = Type IV-NS1 protoein
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTA

SEQ ID NO: 9 - protein, 352 aa, Zika virus
  source 1..352: note = NS1 protein
GCSVDFSKKETRCGTGVFVYNDVEAWRDRYKYHPDSPRRLAAAVKQAWEEGICGISSVSRMENIMWKSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAAHSDLGYWIESEKNDTWRLKRAHLIEMKTCEWPKSHTLWTDGVEESDLIIPKSLAGPLSHHNTREGYRTQVKGPWHSEELEIRFEECPGTKVHVEETCGTRGPSLRSTTASGRVIEEWCCRECTMPPLSFRAKDGCWYGMEIRPRKEPESNLVRSMVTAGS

SEQ ID NO: 10 - protein, 406 aa, Zika virus
  source 1..406: note = Envelope protein
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGST

SEQ ID NO: 11 - protein, 352 aa, Zika virus
  source 1..352: note = NS1 protein
GCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGS

SEQ ID NO: 12 - protein, 402 aa, West Nile virus
  source 1..402: note = Envelope protein
WVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAAL

SEQ ID NO: 13 - protein, 352 aa, West Nile virus
  source 1..352: note = NS1 protein
DTGCAIDISRQELRCGSGVFIHNDVEAWMDRYKYYPETPQGLAKIIQKAHKEGVCGLRSVSRLEHQMWEAVKDELNTLLKENGVDLSVVVEKQEGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNA

SEQ ID NO: 14 - protein, 400 aa, Japanese encephalitis virus
  source 1..400: note = Envelope protein
FNCLGMGNRDFIEGASGATWVDLVLEGDSCLTIMANDKPTLDVRMINIEASQLAEVRSYCYHASVTDISTVARCPTTGEAHNEKRADSSYVCKQGFTDRGWGNGCGLFGKGSIDTCAKFSCTSKAIGRTIQPENIKYEVGIFVHGTTTSENHGNYSAQVGASQAAKFTVTPNAPSITLKLGDYGEVTLDCEPRSGLNTEAFYVMTVGSKSFLVHREWFHDLALPWTSPSSTAWRNRELLMEFEGAHATKQSVVALGSQEGGLHQALAGAIVVEYSSSVKLTSGHLKCRLKMDKLALKGTTYGMCTEKFSFAKNPVDTGHGTVVIELSYSGSDGPCKIPIVSVASLNDMTPVGRLVTVNPFVATSSANSKVLVEMEPPFGDSYIVVGRGDKQINHHWHKAG

SEQ ID NO: 15 - protein, 352 aa, Japanese encephalitis virus
  source 1..352: note = NS1 protein
DTGCAIDITRKEMRCGSGIFVHNDVEAWVDRYKYLPETPRSLAKIVHKAHKEGVCGVRSVTRLEHQMWEAVRDELNVLLKENAVDLSVVVNKPVGRYRSAPKRLSMTQEKFEMGWKAWGKSILFAPELANSTFVVDGPETKECPDEHRAWNSMQIEDFGFGITSTRVWLKIREESTDECDGAIIGTAVKGHVAVHSDLSYWIESRYNDTWKLERAVFGEVKSCTWPETHTLWGDDVEESELIIPHTIAGPKSKHNRREGYKTQNQGPWDENGIVLDFDYCPGTKVTITEDCSKRGPSVRTTTDSGKLITDWCCRSCSLPPLRFRTENGCWYGMEIRPVMHDETTLVRSQVDA

SEQ ID NO: 16 - protein, 352 aa, Tick-borne Encephalitis virus
  source 1..352: note = NS1 protein
DVGCAVDTERMELRCGEGLVVWREVSEWYDNYAYYPETPGALASAIKETFEEGSCGVVPQNRLEMAMWRSSVTELNLALAEGEANLTVVVDKFDPTDYRGGVPGLLKKGKDIKVSWKSWGHSMIWSIPEAPRRFMVGTEGQSECPLERRKTGVFTVAEFGVGLRTKVFLDFRQEPTHECDTGVMGAAVKNGMAIHTDQSLWMRSMKNDTGTYIVELLVTDLRNCSWPASHTIDNADVVDSELFLPASLAGPRSWYNRIPGYSEQVKGPWKYTPIRVIREECPGTTVTINAKCDKRGASVRSTTESGKVIPEWCCRACTMPPVTFRTGTDCWYAMEIRPVHDQGGLVRSMVVA

SEQ ID NO: 17 - protein, 352 aa, Yellow fever virus
  source 1..352: note = NS1 protein
DQGCAINFGKRELKCGDGIFIFRDSDDWLNKYSYYPEDPVKLASIVKASFEEGKCGLNSVDSLEHEMWRSRADEINAIFEENEVDISVVVQDPKNVYQRGTHPFSRIRDGLQYGWKTWGKNLVFSPGRKNGSFIIDGKSRKECPFSNRVWNSFQIEEFGTGVFTTRVYMDAVFEYTIDCDGSILGAAVNGKKSAHGSPTFWMGSHEVNGTWMIHTLEALDYKECEWPLTHTIGTSVEESEMFMPRSIGGPVSSHNHIPGYKVQTNGPWMQVPLEVKREACPGTSVIIDGNCDGRGKSTRSTTDSGKVIPEWCCRSCTMPPVSFHGSDGCWYPMEIRPRKTHESHLVRSWVTA